(19) United States
(12) Patent Application Publication
(10) Pub. No.: US 2024/0066071 A1
(43) Pub. Date: Feb. 29, 2024

(54) SECRETOME FRACTIONS AND USES THEREOF

(71) Applicant: NOVEOME BIOTHERAPEUTICS, INC., Pittsburgh, PA (US)

(72) Inventors: Howard C. WESSEL, Sarver, PA (US); Larry R. BROWN, Newton, MA (US); Ziv Z. KIRSHNER, Monroeville, PA (US)

(21) Appl. No.: 18/036,815

(22) PCT Filed: Nov. 16, 2021
(86) PCT No.: PCT/US2021/059521

§ 371 Date: May 12, 2023

Related U.S. Application Data
(60) Provisional application No. 63/114,060, filed on Nov. 16, 2020.

(51) Int. Cl. A61K 35/50; C12N 5/073; A61P 25/00

(52) U.S. Cl. CPC A61K 35/50; C12N 5/0605; A61P 25/00

(57) ABSTRACT

Molecular weight fractions of a conditioned medium of cultured amnion-derived multipotent progenitor cells, i.e., secretome fractions, and methods of processing a secretome such that it is separated into molecular weight fractions. Uses of the molecular weight secretome fractions to stimulate Schwann cell proliferation, promote neuroprotection, reduce retinal ganglion cell (RGC) loss, and reduce optic nerve inflammation in patients in need thereof.

### RELATED APPLICATION

The present application claims the benefit under 35 U.S.C. § 119(e) of prior U.S. Provisional Application Ser. No. 63/114,060, filed Nov. 16, 2020, the content of which is incorporated herein in its entirety.

### TECHNICAL FIELD

The present disclosure is directed to secretome fractions of defined molecular weights, methods of processing a secretome into defined molecular weight fractions, and therapeutic uses of such molecular weight fractions.

### BACKGROUND

A secretome is the secreted products of a cell. The secretome contains proteins, lipids, nucleic acids, etc., as well as fragments of such molecules, which may or may not have biological activity. In vivo, cells secrete their products into the extracellular space. In vitro, cells secrete their products into culture media, so the secretome is a mixture of the secreted products and the culture media components unless steps are taken to separate the two. The secretome and culture media mixture is often referred to as conditioned media. Many studies have looked at the biological activity of conditioned media in various in vitro and in vivo model systems. A more difficult task is to determine which of the myriad components in the conditioned media is attributable to which biological activities. Numerous technologies exist to fractionate complex mixtures such as conditioned media by various criteria, e.g., molecular weight, isoelectric point, etc. What is not obvious is what, if any, biological activity(ies) will be present or absent in any given fraction or that removal of a fraction may result in enhanced biologic activity.

Applicant has discovered and developed a conditioned medium produced by culturing amnion-derived multipotent progenitor cells. This novel amnion-derived cellular cytokine solution (ACCS), also referred to herein as ST266, contains hundreds of cytokines and growth factors as well as other biologically and non-biologically active molecules. These components are present at physiological concentrations, typically pg/mL to ng/mL levels (Steed, et al., ePlasty Vol. 8, pgs. 157-165, published Apr. 14, 2008). ST266 has been shown to possess anti-inflammatory activity, to be neuroprotective and reduce demyelination (Khan, et al., Scientific Reports Vol. 7, pgs. 41768, 2017), to be anti-apoptotic (U.S. Pat. No. 9,574,177), and to promote impaired wound healing (Wayne, et al., World J Surgery Vol. 34(7), pgs. 1663-8, 2010).

Due to the complex nature of ST266, it has been difficult to understand all of the potential biological and non-biological activities it may possess, and in what mechanisms of action they may be involved. For example, ST266 is known to upregulate the pAKt pathway, which reduces oxidative stress, reduces apoptosis, and enhances neuronal survival via the PI3 kinase pathway. It has also been shown to up-regulate the SIRT-1 pathway, which promotes cell stress responses and survival and upregulates mitochondrial biogenesis. It has also been shown to down-regulate mitochondrial oxidation. (Khan, et al., Scientific Reports 7, 41768, 2017).

Isolation of certain of these activities into specific fractions of the ST266 may allow targeting of individual pathways and more directed therapeutic uses for the ST266 fractions.

### BRIEF SUMMARY

To further understand the nature of the biological activities and what molecular fractions of ST266 may contain these and other activities, Applicant has fractionated the novel secretome ST266 to create distinct molecular weight fractions. These fractions were tested in established models in which ST266 has known activity.

Accordingly, an aspect of the present disclosure includes select molecular weight fractions of the ST266. The fractions include a <30 kDa molecular weight fraction of ST266, a <50 kDa molecular weight fraction of ST266, a 30 kDa-50 kDa molecular weight fraction of ST266, a >30 kDa molecular weight fraction of ST266, and a >50 kDa molecular weight fraction of ST266.

Another aspect of the present disclosure is a method of making <30 kDa and >30 kDa molecular weight fractions of ST266, the method comprising the steps of placing ST266 in a top chamber of an ultrafiltration unit having a filtration membrane with a molecular weight cutoff of 30 kDa, centrifuging the ultrafiltration unit containing the ST266 at 4000 g at 4° C. such that the <30 kDa ST266 components pass through the 30 kDa cutoff filtration membrane, and collecting the <30 kDa fraction in the bottom chamber of the ultrafiltration unit, and the >30 kDa fraction in the top chamber of the ultrafiltration unit.

Another aspect of the present disclosure is a <30 kDa molecular weight fraction of ST266 and/or a >30 kDa molecular weight fraction of ST266 made by the method(s) disclosed herein.

Another aspect of the present disclosure is a method of making <50 kDa and >50 kDa molecular weight fractions of ST266, the method comprising the steps of placing ST266 in a top chamber of an ultrafiltration unit having a filtration membrane with a molecular weight cutoff of 50 kDa, centrifuging the ultrafiltration unit containing the ST266 at 4000 g at 4° C. such that the <50 kDa ST266 components pass through the 30 kDa cutoff filtration membrane, and collecting the <50 kDa fraction in the bottom chamber of the ultrafiltration unit, and the >50 kDa fraction in the top chamber of the ultrafiltration unit.

Another aspect of the present disclosure is a <50 kDa molecular weight fraction of ST266 and/or a >50 kDa molecular weight fraction of ST266 made by the method(s) disclosed herein.

Another aspect of the present disclosure is a method of making a 30 kDa-kDa molecular weight fraction of ST266, the method comprising the steps of placing ST266 in a top chamber of an ultrafiltration unit having a filtration membrane with a molecular cutoff of 30 kDa, centrifuging the ultrafiltration unit containing the ST266 at 4000 g at 4° C. such that the <30 kDa ST266 components pass through the 30 kDa cutoff filtration membrane, collecting the >30 kDa fraction in the top chamber of the ultrafiltration unit, placing the >30 kDa fraction in a top chamber of an ultrafiltration unit having a filtration membrane with a molecular cutoff of 50 kDa, centrifuging the ultrafiltration unit containing the >30 kDa fraction at 4000 g at 4° C. such that the <50 kDa ST266 components pass through the 50 kDa cutoff filtration membrane, collecting the 30 kDa-50 kDa fraction in the bottom chamber of the ultrafiltration unit.

Another aspect of the present disclosure is a method of making a 30 kDa-kDa molecular weight fraction of ST266, the method comprising the steps of placing ST266 in a top chamber of an ultrafiltration unit having a filtration membrane with a molecular cutoff of 50 kDa, centrifuging the ultrafiltration unit containing the ST266 at 4000 g at 4° C. such that the <50 kDa ST266 components pass through the 50 kDa cutoff filtration membrane, collecting the <50 kDa fraction in the bottom chamber of the ultrafiltration unit, placing the <50 kDa fraction in a top chamber of an ultrafiltration unit having a filtration membrane with a molecular cutoff of 30 kDa, centrifuging the ultrafiltration unit containing the <50 kDa fraction at 4000 g at 4° C. such that the <30 kDa ST266 components pass through the 30 kDa cutoff filtration membrane, collecting the 30 kDa-50 kDa fraction in the top chamber of the ultrafiltration unit.

Another aspect of the present disclosure is a method of stimulating Schwann cell proliferation in a patient in need thereof comprising the step of administering to the patient a <30 kDa molecular weight fraction of ST266.

Another aspect of the present disclosure is a method for promoting neuroprotection is a patient in need thereof comprising the step of administering to the patient a <30 kDa fraction of ST266.

Another aspect of the present disclosure is a method of stimulating Schwann cell proliferation in a patient in need thereof comprising the step of administering to the patient a <50 kDa molecular weight fraction of ST266.

Another aspect of the present disclosure is a method of reducing demyelination in a patient in need thereof comprising the step of administering to the patient a <50 kDa molecular weight fraction of ST266. The demyelination may be in the patient's spinal cord.

Another aspect of the present disclosure is a method for promoting neuroprotection in a patient in need thereof comprising the step of administering to the patient a <50 kDa fraction of ST266.

Another aspect of the present disclosure is a method of reducing retinal ganglion cell (RGC) loss in a patient in need thereof comprising the step of administering to the patient a <50 kDa molecular weight fraction of ST266.

Another aspect of the present disclosure is a method of reducing optic nerve inflammation in a patient in need thereof comprising the step of administering to the patient a <50 kDa molecular weight fraction of ST266.

Another aspect of the present disclosure is a method of stimulating Schwann cell proliferation in a patient in need thereof comprising the step of administering to the patient a >30 kDa or >50 kDa molecular weight fraction of ST266.

Another aspect of the present disclosure is a method of reducing demyelination in a patient in need thereof comprising the step of administering to the patient a >30 kDa or >50 kDa molecular weight fraction of ST266. The demyelination may be in the patient's spinal cord.

Another aspect of the present disclosure is a method for promoting neuroprotection in a patient in need thereof comprising the step of administering to the patient a >30 kDa or >50 kDa fraction of ST266.

Another aspect of the present disclosure is a method of reducing retinal ganglion cell (RGC) loss in a patient in need thereof comprising the step of administering to the patient a >30 kDa or >50 kDa molecular weight fraction of ST266.

Another aspect of the present disclosure is a method of reducing optic nerve inflammation in a patient in need thereof comprising the step of administering to the patient a <50 kDa molecular weight fraction of ST266.

Another aspect of the present disclosure is a method of stimulating Schwann cell proliferation in a patient in need thereof comprising the step of administering to the patient a 30 kDa-50 kDa molecular weight fraction of ST266.

Another aspect of the present disclosure is a method of reducing demyelination in a patient in need thereof comprising the step of administering to the patient a 30 kDa-50 kDa molecular weight fraction of ST266. The demyelination may be in the patient's spinal cord.

Another aspect of the present disclosure is a method for promoting neuroprotection in a patient in need thereof comprising the step of administering to the patient a 30 kDa-50 kDa fraction of ST266.

Another aspect of the present disclosure is a method of reducing retinal ganglion cell (RGC) loss in a patient in need thereof comprising the step of administering to the patient a 30 kDa-50 kDa molecular weight fraction of ST266.

Another aspect of the present disclosure is a method of reducing optic nerve inflammation in a patient in need thereof comprising the step of administering to the patient a <50 kDa molecular weight fraction of ST266.

The various molecular weight fractions described herein may be administered in a therapeutically effective amount that may depend on the disease or disorder for which it is being administered.

The various molecular weight fractions described herein may be administered to the patient by a route selected from the group consisting of targeted intranasal administration and systemic administration. Exemplary systemic administration is selected from the group consisting of intravenous administration and intraperitoneal administration.

### Definitions and Abbreviations

As defined herein “isolated” refers to material removed from its original environment and is thus altered “by the hand of man” from its natural state.

As used herein, the term “Amnion-derived Multipotent Progenitor cell” or “AMP cell” means a specific population of cells that are epithelial cells derived from the amnion of a placenta. AMP cells secrete a unique combination of physiologically relevant cytokines in a physiologically relevant temporal manner into the extracellular space or into surrounding culture media. AMP cells have not been cultured in the presence of any non-human animal-derived products, making them and cell products derived from them suitable for human clinical use. In a preferred embodiment, the AMP cells secrete the cytokines VEGF, Angiogenin, PDGF, and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜0.68 μg/mL for TIMP-1, and ˜1.04 μg/mL for TIMP-2. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, have no reaction with an antibody to the stem/progenitor cell marker c-kit (CD117), and minimal to no reaction with an antibody to the stem/progenitor cell marker Thy-1 (CD90).

By the term “animal-free” when referring to certain compositions, growth conditions, culture media, etc., described herein, is meant that no non-human animal-derived materials, such as bovine serum, proteins, lipids, carbohydrates, nucleic acids, vitamins, etc., are used in the preparation, growth, culturing, expansion, storage or formulation of AMP cells and their secreted product ST266, composition, or process. By “no non-human animal-derived materials” is meant that the materials have never been in or in contact with a non-human animal body or substance, so they are not xeno-contaminated. Only clinical grade materials, such as recombinantly produced human proteins, are used in the preparation, growth, culturing, expansion, storage, and/or formulation of AMP cells and ST266.

By the term “serum-free” is meant that no non-human animal-derived serum is used in the preparation, growth, culturing, expansion, storage, or formulation of AMP cells and their secreted product ST266.

As used herein, “conditioned medium” is a medium in which a specific cell or population of cells has been cultured, and then the cells are removed from the medium. When cells are cultured in a medium, they secrete cellular factors that can provide support to or affect the behavior of other cells. Such factors include, but are not limited to hormones, cytokines, extracellular matrix (ECM), proteins, vesicles, antibodies, chemokines, receptors, inhibitors, and granules. The medium containing the cellular factors is the conditioned medium.

As used herein, the term “ST266” (previously termed “Amnion-derived Cellular Cytokine Solution” or “ACCS”) means conditioned medium that has been made by culturing AMP cells.

The term “physiologic” or “physiological level” as used herein means the level that a substance in a living system is found and that is relevant to the proper functioning of a cellular, biochemical and/or biological process.

As used herein, the term “secretome” means the secreted products of a cell. A secretome may contain proteins, lipids, nucleic acids, etc., as well as fragments of such molecules, which may or may not have biological activity.

As used herein, the term “fractionation” means the process of separating a complex biological mixture into distinct fractions based on molecular weight, salt fractionation, isoelectric point, and the like.

As used herein, the term “therapeutically effective amount” means that amount of a therapeutic agent necessary to achieve a desired physiological effect (i.e., stimulate Schwann cell proliferation, reduce RGC loss, reduce demyelination).

As used herein, the term “pharmaceutically acceptable” means that the components, in addition to the therapeutic agent, comprising the formulation, are suitable for administration to the patient being treated in accordance with the present disclosure.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

As used herein, the term “tissue” refers to an aggregation of similarly specialized cells united in the performance of a particular function.

It must be noted that as used herein and in the appended claims, the singular forms “a,” “and” and “the” include plural references unless the context clearly dictates otherwise.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time release capsules and the like.

The terms “parenteral administration” and “administered parenterally” are art-recognized and refer to modes of administration other than nasal, targeted intranasal, transcribriform nose-to-brain, enteral, and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral, intraocular, intracameral, subdural, and intrasternal injection or infusion.

As used herein, the term “enteral” administration means any route of drug administration that involves absorption of the drug through the gastrointestinal tract. Enteral administration may be divided into three different categories: oral, gastric, and rectal.

As used herein, the term “topical” administration means a medication that is applied to body surfaces such as the skin, eye, or mucous membranes to treat ailments using a large range of formulations including, but not limited to, liquids, sprays, creams, foams, gels, lotions, salves, powders and ointments.

The term “intranasal” or “intranasal delivery” or “intranasal administration” or “targeted intranasal” as used herein means delivery through the nasal cavity to the olfactory epithelium adjacent to the cribriform plate. Such administration utilizes a targeted intranasal delivery device, including transcribriform direct nose-to-brain.

The terms “sustained-release”, “extended-release”, “time-release”, “controlled-release”, or “continuous-release” as used herein means an agent, typically a therapeutic agent or drug, that is formulated to dissolve slowly and be released over time.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein the term “standard animal model” refers to any art-accepted animal model in which the compositions of the invention exhibit efficacy.

The word “comprising” and forms of the word “comprising”, as used in this description and in the claims, does not limit the present invention to exclude any variants or additions. Additionally, although the present invention has been described in terms of “comprising”, the processes, materials, and compositions detailed herein may also be described as consisting essentially of or consisting of. For example, while certain aspects of the invention have been described in terms of a method comprising administering an effective amount of a <30 kDa molecular weight fraction of ST266, a method “consisting essentially of” or “consisting of” administering an effective amount of a <30 kDa molecular weight fraction of ST266 is also within the present scope. In this context, “consisting essentially of” means that any additional components will not materially affect the efficacy of the method.

“Therapeutically effective amount” or “effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in demyelination, increased neuroprotection, reduced RGC loss, reduced optic nerve inflammation, and the like. According to certain aspects, “therapeutically effective amount” or “effective amount” refers to an amount of the ST266 fraction that cause one or more of reduction in demyelination, increased neuroprotection, reduced RGC loss, and/or reduced optic nerve inflammation.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Moreover, other than in the examples, or where otherwise indicated, all numbers expressing, for example, quantities of ingredients used in the specification are to be understood as being modified in all instances by the term “about”. At the very least, and not as an attempt to limit the application of the doctrine of equivalents to the scope of the claims, each numerical parameter should at least be construed in light of the number of reported significant digits and by applying ordinary rounding techniques. Thus, the term “about” when used before a numerical designation, e.g., temperature, time, amount, and concentration, including a range, indicates approximations which may vary by ±10%, ±5%, or ±1%.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

### DETAILED DESCRIPTION

Disclosed herein are specific molecular weight fractions of ST266, and therapeutic methods of use of those fractions. Fractionation of the ST266 may isolate certain activities and components, and/or may target specific biological pathways in which those components participate and may thus allow for more directed therapeutic uses of the ST266 fractions.

Compositions and Methods of Making ST266 Compositions

Detailed information and methods on the preparation of AMP cell compositions, generation of ST266 (formerly ACCS) can be found in U.S. Pat. Nos. 8,058,066; 8,088,732; and 8,278,095, all of which are incorporated herein by reference.

ST266 is a composition comprising the proteins VEGF, Angiogenin, PDGF, TGFβ2, TIMP-1 and TIMP-2, wherein the proteins levels are ˜5.0-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2.

ST266 is made by a method comprising the steps of:
a) obtaining a placenta and isolating the amnion from the placenta,
b) enzymatically releasing amnion-derived epithelial cells from the amnion,
c) collecting the released amnion-derived epithelial cells, and
d) culturing the collected amnion-derived epithelial cells of step (c) in a tissue culture vessel for about 2 days in Iscove's Modified Dulbecco's Media (IMDM) culture medium that does not contain any non-human animal protein and is, supplemented with 5 mg/mL recombinant human serum albumin and 15 ng/mL recombinant human EGF,
e) selecting amnion-derived multipotent progenitor (AMP) cells from the amnion-derived epithelial cells of step (d) by removing and discarding the culture medium which contains cells that have not attached to the tissue culture vessel and keeping the cells that have attached to the tissue culture vessel, such attached cells consisting essentially of AMP cells,
f) culturing the selected AMP cells of step (e) in IMDM culture medium that does not contain any non-human animal protein and is supplemented with 5 mg/mL recombinant human serum albumin and 15 ng/mL recombinant human EGF until they reach confluence,
g) removing the culture medium from the confluent AMP cells in step (f) and culturing the confluent AMP cells with fresh culture medium and culturing the AMP cells for 1, 2, 3, 4, 5, or 6 days,
h) collecting the culture medium of step (g) to obtain ST266 and adding fresh medium to the confluent AMP cells, and
i) repeating steps (g) and (h) a plurality of times and combining the ST266 obtained in each step (h) to create the pooled ST266.

Fractionation Methods

Numerous fractionation methods exist. While Applicant utilized ultracentrifugation to prepare the ST266 fractions described herein, one skilled in the art will recognize that any fractionation method known in the art that can separate full complement ST266 into <30 kDa, >30 kDa, <50 kDa, >50 kDa, and 30 kDa-50 kDa fractions is suitable for use in practicing the invention.

In these studies, fractions of ST266 were prepared using Amicon Ultra-15 filters (MilliporeSigma; 50 kDa Cat #: UFC905024 and 30 kDa Cat #: UFC903024). The centrifuge was set to 4° C. and 4,000 g. Filters were first washed with 15 mL Water for Injection as per manufacture's recommendation. Water was removed, and ST266 was loaded into the filter. All solutions and filter units were used at 4° C. (e.g., ST266, buffers, filter units may be precooled). Material passing through each filter is considered to be less then the molecular weight cutoff of the filter, and material remaining in the upper chamber is considered to be above the molecular weight cutoff of the filter. The volumes and spin times for each filter were selected based on known procedures and as indicated in the user guide for each filter unit.

Therapeutic Applications

The ST266 fractions described herein, i.e., the <30 kDa, >30 kDa, <50 kDa, >50 kDa, and 30 kDa-50 kDa fractions, have therapeutic utility in promoting neuroprotection in a patient in need thereof. Non-limiting specific examples are as follows.

All of the ST266 fractions described herein have therapeutic utility in stimulating Schwann cell proliferation in the nervous system. Schwann cells are responsible for producing the myelin sheath in the nervous system, a structure that protects neuron axons and supports proper transmission of electrical impulses by neurons. In particular, as shown in the examples section, the <30 kDa and <50 kDa fractions have been found to stimulate Schwann cell proliferation in the nervous system.

Certain of the ST266 fractions described herein, i.e., the >30 kDa, <50 kDa, >50 kDa, and 30 kDa-50 kDa fractions, have therapeutic utility in reducing demyelination of neurons, a condition often seen in various demyelinating conditions such as optic neuritis. Optic neuritis is often the presenting symptom of multiple sclerosis and can lead to vision loss and even blindness. In particular, as shown in the examples section, the <50 kDa fraction described herein, has therapeutic utility in reducing demyelination of neurons.

Certain of the ST266 fractions described herein, i.e., the >30 kDa, <50 kDa, >50 kDa, and 30 kDa-50 kDa fractions, have therapeutic utility in reducing retinal cell ganglion cell (RGC) loss. RGCs are one of five cell types that make up the retina. Their axons leave the retina at the optic head and, bundled together, form the optic nerve. The optic nerve is responsible for carrying sensory information from the eyes to the brain. Optic neuritis is often the presenting symptom of multiple sclerosis and can lead to vision loss and even blindness. Loss of RGCs results in loss of vision, and even blindness. In particular, as shown in the examples section, the <50 kDa fraction described herein, has therapeutic utility in reducing retinal cell ganglion cell (RGC) loss.

Certain of the ST266 fractions described herein, i.e., the >30 kDa, <50 kDa, >50 kDa, and 30 kDa-50 kDa fractions, have therapeutic utility in reducing inflammation of the optic nerve. Inflammation of this nerve leads to demyelination and RGC loss, leading to vision loss and blindness. In particular, as shown in the examples section, the <50 kDa fraction described herein, has therapeutic utility in reducing inflammation of the optic nerve. Thus, diseases of the optic nerve, such as optic neuritis, as well as injuries to the optic nerve, such as crush injuries, can be treated with the <50 kDa fraction of ST266.

It is contemplated that the ST266 fractions will be useful in reducing inflammation and demyelination and stimulating cell proliferation in other therapeutic indications, as well.

Formulation

Compositions comprising the ST266 fractions described herein may be administered to a subject in need. Such compositions may be formulated in any conventional manner using one or more physiologically acceptable carriers optionally comprising excipients and auxiliaries. Proper formulation is dependent upon the route of administration chosen. For topical administration, the ST266 fractions may be formulated as a spray, liquid, cream, foam, gel, lotion, salve, powder and ointment, etc. The compositions may also be administered to the recipient in one or more physiologically acceptable carriers. Carriers for the ST266 fractions may include, but are not limited to, solutions of normal saline, phosphate buffered saline (PBS), lactated Ringer's solution containing a mixture of salts in physiologic concentrations, or cell culture medium.

Administration

For parenteral administration, the formulation may be injected intravenously, although other routes of parenteral administration are contemplated by the instant invention.

For enteral administration, the formulation may be administered as a liquid, a capsule or tablet that can be administered orally, rectally or gastrically.

For subcutaneous or intramuscular administration, the formulation may be delivered by needle and syringe, by pen injectors, by needle-less injection devices, and the like.

For nasal administration, the formulation may be administered as a nasal spray, a nebulized pulmonary dosage form, a metered dose inhaler or a dry powder inhaler.

For targeted intranasal administration the formulation is administered using a targeting delivery device.

In addition, one of skill in the art may readily determine the appropriate dose of the <30 kDa and <50 kDa fractions of ST266 for a particular purpose.

Dose

Exemplary intravenous doses may range from about 0.01 mL/kg to 100 mL/kg. In a preferred embodiment, a preferred range of the ST266 fractions is 0.5-1.0 mL/kg once or twice a day. One of skill in the art will recognize that the number of doses to be administered needs also to be empirically determined based on, for example, severity and type of disease, disorder or injury being treated; patient age, weight, gender, health status; other medications and treatments being administered to the patient; and the like. For example, in a specific embodiment, one dose is sufficient to have a therapeutic effect. Other specific embodiments contemplate, 2, 3, 4, or more doses for therapeutic effect.

Exemplary targeted intranasal nose-to-brain administration doses may range from 200 μL in one nostril once a day for multiple days to 400 μL in both nostrils twice a day for multiple days. Dose and dosing schedule will be determined based on the condition being treated. For example, acute conditions may require frequent dosing for a shorter number of days while chronic conditions may require less frequent doing over several days to months or longer.

Exemplary topical doses include a dose is in the range of about 0.1-to-1000 μg/cm² of applied area. Other preferred dose ranges are 1.0-to-50.0 μg/cm² applied area.

One of skill in the art will also recognize that number of doses (dosing regimen) to be administered will need often need to be empirically determined based on, for example, the patient age, weight, sex, health; other medications and treatments being administered to the patient; acute versus chronic conditions; and the like.

Additional Agents

In further embodiments of the present invention, at least one additional agent may be combined with the <30 kDa or <50 kDa fractions. Such agents may act synergistically with the fractions to enhance the therapeutic effect. Such agents include, but are not limited to, growth factors, cytokines, chemokines, antibodies, inhibitors, antibiotics, immunosuppressive agents, steroids, anti-fungals, anti-virals or cells. Inactive agents include carriers, diluents, stabilizers, gelling agents, delivery vehicles, ECMs (natural and synthetic), scaffolds, matrices and the like. When the ST266 fractions are administered conjointly with other pharmaceutically active agents, even less of the fractions may be needed to be therapeutically effective.

### EXAMPLES

The following examples are put forth to provide those of ordinary skill in the art with a complete disclosure and description of how to make and use the compositions and methods of the present invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celsius, and pressure is at or near atmospheric.

### Example 1: Fractionation of ST266

Fractions of ST266 were prepared using Amicon Ultra-15 filters (MilliporeSigma; 50 kDa Cat #: UFC905024 and 30 kDa Cat #: UFC903024). The centrifuge was precooled to 4° C. and filtrates were obtained on the 30 kDa and 50 kDA filters by centrifugation at 4° C. and 4,000 g. For each filter, depending on the molecular weight cut off, spin time was optimized in earlier experiments. Filters were first washed with 15 mL Water for Injection (WFI) as per manufacture's recommendation. Water was removed, and ST266 was loaded into the filter.

### Example 2: Schwann Cell Proliferation Assay

Method: Schwann cell proliferation assays were performed to assess the ability of the ST266 fractions to stimulate proliferation of these cells. SW10 Mouse Schwann Cells (ATCC Cat #CRL-2766) were seeded in 96-well tissue culture treated plates and cultured in normal growth media (NGM), containing Dulbecco's Modified Eagle Medium (DMEM; Gibco, Cat #11054-020), GlutaMAX (Gibco, Cat #35050061) and 10% FBS (GE Healthcare, HyClone Cat #SH30071.01) for a fixed amount of time at 37° C. and 5% CO₂. When acclimated, NGM media was replaced by minimal starvation medium for 24-hours and subsequently replaced by one of the treatment medias: NGM, Iscove's Modified Dulbecco's Media (IMDM; growth under minimal conditions), STM100 (proprietary base medium for culturing AMP cells), ST266 or a filtrate fraction of ST266 below the molecular weight cut-off of either 50 kDa (<50 kDa) or 30 kDa (<30 kDa).

After 24-hours, Schwann cell proliferation was determined using a commercially available viable cell counting kit (Sigma-Aldrich, Cat #96992). This colorimetric assay measures the water-soluble formazan dye formed by NADH-mediated reductions in extracellular tetrazolium salt WST-8. Absorbance was read at 450 nm using Synergy 2 plate reader (Bio Tek) and either the STM100 or NGM blank media signal was subtracted. For each plate, proliferation was normalized to NGM treatment and data reported as a ratio of sample absorbance to the NGM value. Experiments were repeated three times, each with freshly made ST266 filtrate fractions.

Results: ST266 increases Schwann cell proliferation following starvation conditions more potently than either <50 kDa or <30 kDa filtrate fractions.

One-way ANOVA with six groups revealed significant differences in Schwann cell proliferation between treatments (p<0.0001). Tukey's post-hoc analysis revealed that Schwann cell proliferation is significantly higher with treatment of ST266 or either of the ST266 fractions compared to IMDM and STM100 controls (p<0.0001 for all comparisons). The proliferation effects of the <50 kDa and <30 kDa ST266 fractions were significantly lower than non-fractionated ST266 (p=0.032 and p<0.0001, respectively). No significant difference was found between the effect of <50 kDa and <30 kDa fractions (p=0.11, ns), although the <50 kDa fraction showed a trend toward increased proliferation.

### Example 3: Evaluation of ST266 Fractions in the Experimental Autoimmune Encephalomyelitis (EAE) of Multiple Sclerosis and Optic Neuritis Mouse Model

Method: Female C57BL/6J mice were purchased from the Jackson Laboratory (Bar Harbor, ME, USA). All experiments in this study adhered to the Association for Research in Vision and Ophthalmology Statement for the Use of Animals in Ophthalmic and Vision Research and were compliant with the University of Pennsylvania Institutional Animal Care and Use Committee guidelines and policies.

Chronic EAE was induced in mice as previously described (Khan, et al., Scientific Reports Vol. 7, pg. 41768, 2017). Briefly, at age eight weeks, mice were anesthetized with isoflurane and a sub-cutaneous injection was done at two dorsal sites containing a total of 200 μg myelin oligodendrocyte glycoprotein (MOG) (MOG 35-55; Genscript, Piscataway, NJ, USA) emulsified in Complete Freund's Adjuvant (Difco, Detroit, MI, USA) with 2.5 mg/mL killed Mycobacterium tuberculosis (Difco). Control mice were injected with equivalent volumes of PBS accompanied by equal doses of Complete Freund's Adjuvant and M. tuberculosis. Each animal was also injected intraperitoneally with 200 ng pertussis toxin dissolved in 0.1 mL PBS at the time of initial immunization and again 48 hours later. Disease severity based on ascending paralysis was scored daily using previously described scales as follows: no disease=0; partial tail paralysis=0.5; tail paralysis or waddling gait=1.0; partial tail paralysis and waddling gait=1.5; tail paralysis and waddling gait=2.0; partial limb paralysis=2.5; paralysis of one limb=3.0; paralysis of one limb and partial paralysis of another=3.5; paralysis of two limbs=4.0; moribund state=4.5; death=5.0.

Aliquots of ST266, the <30 kDa fraction and the <50 kDa fraction prepared as described above for in vitro Schwann cell proliferation assays were stored at 4° C. For intranasal dosing, unanesthetized mice were secured by the scruff and 20 μL of the test agent or vehicle control were instilled in the nares once daily, similar to prior studies (Khan, et al., 2017).

Optokinetic nystagmus reflex (i.e., optokinetic reflex or OKR) was used to estimate visual performance in mice using the OptoMotry apparatus and software (CerebralMechanics Inc., Medicine Hat, Alberta, CA). Mice were positioned on a platform surrounded on all sides by video monitors displaying 100% contrast sinusoidal black and white bands rotating clockwise or counterclockwise. A trained masked observer graded head movement in the direction of rotation of the bands to detect the threshold spatial frequency (cycles per degree) where an animal fails to track the pattern.

RGCs were quantified by Brn3a immunolabeling using previously described methods. In summary, following euthanasia, eyes were removed and fixed in 4% paraformaldehyde at 4° C. overnight. Retinas were dissected, washed with phosphate buffered saline (PBS) containing 0.5% Triton X-100, and then permeabilized by freezing at −80° C. for ten minutes. After thawing, each retina was labeled with rabbit anti-mouse Brn3a antibody (Synaptic Systems #411003, Goettingen, Germany) at 1:4000 dilution in blocking buffer containing PBS with 2% bovine serum albumin and 2% Triton X-100. After overnight incubation at 4° C., retinas were washed four times in PBS and then incubated for one hour at room temperature with an anti-rabbit secondary antibody conjugated to Alexa Fluor 488 (A21206, Thermo Fisher Scientific, Waltham, MA, USA) at 1:4000 dilution in blocking buffer. After washing in PBS×4, retinas were flat-mounted with four radial relaxing cuts and placed RGC side up on positively charged slides and cover-slipped with vectashield antifade mounting media (Vector Laboratories, Burlingame, CA, USA). Photomicrographs were obtained with an epifluorescent microscope by an investigator masked to treatment groups. Three representative regions were captured in each quadrant—corresponding to one-sixth, three-sixths, and five-sixths of the retinal radius—for a total of twelve photos per retina. Digital images were re-labeled with random codes for masking and RGCs were semi-automatically counted and individually validated by visual inspection and manually corrected as needed using established protocols with ImageJ Fiji open source image analysis software.

Optic nerves were isolated at the time of sacrifice, fixed in 4% paraformaldehyde, embedded in paraffin, and cut into 5 μm thick longitudinal sections. For assessment of inflammation, sections were stained with H&E and examined by light microscopy. Scores were assigned to each sample by a masked observer per prior studies as follows: no infiltration=0; mild cellular infiltration of the optic nerve or optic nerve sheath=1; moderate infiltration=2; severe infiltration=3; massive infiltration=4. To detect demyelination, sections of the optic nerve were stained with luxol fast blue (LFB) and quantified on a 0-3 point relative scale by a masked investigator as in prior studies (4, 16): 0=no demyelination; 1=scattered foci of demyelination; 2=prominent foci of demyelination; and 3=large (confluent) areas of demyelination. The entire length of each optic nerve section was examined.

Evaluation of EAE severity scores and OKR thresholds over time were compared using ANOVA of repeated measures followed by Tukey post-hoc comparisons between each group. RGC counts, final OKR scores, optic nerve inflammation scores, optic nerve demyelination scores, and Schwann cell proliferation levels were compared by one-way ANOVA with Tukey post-hoc comparisons between treatment groups. Where indicated, pairwise comparisons were made using the two tailed Student t-test. All computations were done using Graph Pad Prism (GraphPad Software, San Diego, CA, USA). P values less than 0.05 were considered significant.

Results: ST266 and <50 kDa fractionated ST266 suppress optic nerve demyelination in mice with mild EAE/optic neuritis.

EAE mice (n=8/treatment group) and control (non-EAE) mice (n=6) were monitored daily for development of ascending paralysis and weekly for OKR responses, prior to sacrifice at day 42 post-immunization. EAE induction produced only mild EAE disease which was not altered by daily intranasal treatment with ST266 or <50 kDa ST266 and failed to induce a significant decrease in OKR responses. However, even in this cohort of mice with only mild EAE disease, an overall trend toward loss of RGCs in EAE mice was observed, and analysis of RGC survival by retinal region (central, mid-peripheral, and peripheral) showed a small but significant loss of RGCs induced by EAE in the mid-periphery. This regional RGC loss was significantly prevented by daily treatment with ST266, whereas <50 kDa ST266 treatment only led to a non-significant trend towards increased RGC survival as compared to PBS-treated EAE mice. EAE mice developed significant optic nerve inflammation and demyelination, and treatment with both ST266 and <50 kDa ST266 significantly reduced the level of demyelination compared with PBS-treated EAE mice. Both treatments prevented significant optic nerve inflammation from developing as compared to control, non-EAE mice, but showed only a trend towards reducing inflammation compared to PBS-treated EAE mice.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

1-29. (canceled)

30. A therapeutic method comprising:
administering to a patient a therapeutically effective amount of a composition comprising a molecular weight fraction of a conditioned medium produced by culturing amnion-derived multipotent progenitor cells (ST266),
wherein the molecular weight fraction of the ST266 comprises one or more of: a <30 kDa molecular weight fraction of ST266, a <50 kDa molecular weight fraction of ST266, a 30 kDa-50 kDa molecular weight fraction of ST266, a >30 kDa molecular weight fraction of ST266, and a >50 kDa molecular weight fraction of ST266, and
wherein the therapeutically effective amount stimulates Schwann cell proliferation, promotes neuroprotection, reduces demyelination, reduces retinal ganglion cell (RGC) loss, reduces optic nerve inflammation, or any combination thereof.

31. The method of claim 30, wherein the composition is administered to the patient by a route selected from the group consisting of targeted intranasal administration and systemic administration.

32. The method of claim 31, wherein the systemic administration is selected from the group consisting of intravenous administration and intraperitoneal administration.

33. The method according to claim 30, wherein the molecular weight fraction of the ST266 comprises the <50 kDa molecular weight fraction of ST266 and the therapeutically effective amount stimulates Schwann cell proliferation.

34. The method according to claim 33, wherein the molecular weight fraction of the ST266 comprises the >50 kDa molecular weight fraction of ST266 and the therapeutically effective amount reduces retinal ganglion cell (RGC) loss.

35. The method according to claim 34, wherein the composition is administered to the patient by targeted intranasal administration.

36. The method according to claim 30, wherein the molecular weight fraction of the ST266 comprises the <50 kDa molecular weight fraction of ST266 and the therapeutically effective amount reduces optic nerve inflammation.

37. The method according to claim 36, wherein the composition is administered to the patient by targeted intranasal administration.

38. The method according to claim 30, wherein the molecular weight fraction of the ST266 comprises the <30 kDa molecular weight fraction of ST266 and the therapeutically effective amount promotes neuroprotection.

39. The method according to claim 30, wherein the molecular weight fraction of the ST266 comprises the <50 kDa molecular weight fraction of ST266 and the therapeutically effective amount promotes neuroprotection.

40. The method according to claim 30, wherein the molecular weight fraction of the ST266 comprises the >50 kDa molecular weight fraction of ST266 and the therapeutically effective amount promotes neuroprotection.

41. The method according to claim 30, wherein the molecular weight fraction of the ST266 comprises the <50 kDa molecular weight fraction of ST266 and the therapeutically effective amount reduces demyelination.

42. The method according to claim 41, wherein the composition is administered to the patient by targeted intranasal administration.

43. The method according to claim 30, wherein the molecular weight fraction of the ST266 comprises the >30 kDa molecular weight fraction of ST266 and the therapeutically effective amount reduces demyelination.

44. The method according to claim 43, wherein the composition is administered to the patient by targeted intranasal administration.

45. The method according to claim 43, wherein the demyelination is in the patient's spinal cord.

46. The method according to claim 30, wherein the molecular weight fraction of the ST266 comprises the >50 kDa molecular weight fraction of ST266 and the therapeutically effective amount reduces demyelination.

47. The method according to claim 46, wherein the composition is administered to the patient by targeted intranasal administration.

48. The method of claim 46, wherein the demyelination is in the patient's spinal cord.

49. The method according to claim 30, wherein the molecular weight fraction of the ST266 comprises the >50 kDa molecular weight fraction of ST266 and the therapeutically effective amount stimulates Schwann cell proliferation, reduces optic nerve inflammation, promotes neuroprotection, reduces demyelination, or any combination thereof.
